(19) United States
(12) Patent Application Publication
(10) Pub. No.: US 2023/0144805 A1
(43) Pub. Date: May 11, 2023

(54) Methods of Noninvasive Detection and Specific Treatment for Cancer Metastasis

(71) Applicant: Primo Biotechnology Co., Ltd, Taipei City (TW)

(72) Inventors: Ya-Yao Huang, Taipei City (TW); Bing-Ying Ho, Taipei City (TW); Ya-Ting Huang, Taipei City (TW)

(73) Assignee: Primo Biotechnology Co., Ltd, Taipei City, TW (TW)

(21) Appl. No.: 18/054,149

(22) Filed: Nov. 10, 2022

Related U.S. Application Data
(60) Provisional application No. 63/277,615, filed on Nov. 10, 2021.

(51) Int. Cl. G01N 33/534; A61P 35/00; A61K 51/10

(52) U.S. Cl. CPC G01N 33/534; A61P 35/00; A61K 51/10

(57) ABSTRACT

A noninvasive detection method for cancer metastases is provided, which are based on the feature of the radiopharmaceutical that binds specifically to cells of cancer hepatic metastasis, so that cancer metastases can be detected by detecting the signal of the radiopharmaceutical in various organs in the subject. A specific treating method for cancer metastases by using the radiopharmaceutical as an important tool for precise treatment is also provided.

### CROSS-REFERENCE TO RELATED APPLICATION

This application claims the benefit of U.S. Provisional Patent Application No. 63/277,615 filed on Nov. 10, 2021, which is incorporated by reference herein in its entirety.

### FIELD OF THE INVENTION

The present invention provides a noninvasive detection and treatment method applied to cancer metastasis.

### BACKGROUND OF THE INVENTION

The principal cause of cancer lethality is generally considered to be cancer metastases, which are processes that cancer cells disseminate from a primary lesion to distal organs. The new generated tumors in distal organs are called a secondary or metastatic tumors, they are similar to the primary tumor.

Hepatic metastasis is one type of cancer metastases that is widely known. Hepatic metastases refer to the spread of cancer cells from the primary tumor to the liver to form new tumors. Many cancers will have liver metastases, for example, breast cancer, colorectal cancer, lung cancer, etc. Asymptomatic cancers in the early stage tend to be found until late stages, causing high probability of hepatic metastases; while skin cancer and prostate cancer are usually detected early and removed, as a result, the probability of hepatic metastases is low.

Liver is one of the most common sites of cancer metastases, accounting for nearly 25% of all cases. Various primary tumors may be the sources of metastases. Taking colorectal adenocarcinoma as an example, statistics show that about 70-80% of metastatic diseases occurs in the liver. In recent years, about 20-25% of colorectal cancer patients suffers from liver metastases, which shows that liver metastases demand special attentions.

Liver metastases are closely related to the structure of the liver. There are mainly two blood vessels that support the operation of the liver, namely the hepatic artery and the hepatic portal vein. Cancerous cells in different tissues or organs can flow into the liver through these two blood vessels, causing liver lesions which in turn enhance hepatic metastases.

CT imaging is the current detection method which serves as an evaluation method. The location, size and disease progression of metastatic hepatoma can be determined, even the remaining size after liver resection can be estimated by CT scan, which is a mainstream medical model.

For metastatic breast cancer, about 40-60% of the patients will suffer from hepatic metastases. Liver metastases from breast cancer (LMBC) has long been regarded as a systemic disease, which can only be treated with breast cancer chemotherapy drugs and the best supportive care, rather than general surgery. In addition, LMBC status is regarded as one of important prognostic factors of breast cancer.

The present invention aims to provide a method for detecting cancer metastases or for specific treating cancer metastases, comprising using a currently used radiopharmaceutical to combine with a specific antigen exhibited by metastases.

### BRIEF DESCRIPTION OF THE DRAWINGS

FIG. 1(A) is an anatomical diagram of the location of an implanted tumor in a 4T1-tumor mouse of the present invention.

FIG. 1(B) is an image of the 4T1-tumor mouse of the present invention in different organs of in vivo image system (IVIS) images.

FIG. 2 is a panel of images of the [¹⁸F]FDG micro-positron/CT tomography of the present invention.

FIG. 3 is a panel of images by using the ¹⁸F-PSMA-1007 micro-positron/CT scan.

### DETAILED DESCRIPTION OF THE INVENTION

The present invention is a method for early detecting cancer metastases by using a radiopharmaceutical, comprising the steps of administering a radiopharmaceutical to a subject; and detecting the signal of the radiopharmaceutical in various organs in the subject by an instrument, when a signal of the radiopharmaceutical is detected in an organ indicates that the organ of the subject has early cancer metastases.

In one embodiment of the present invention, wherein the cancer metastases are cancer hepatic metastases.

In one embodiment of the present invention, wherein the detected signal of the radiopharmaceutical is a radiation signal.

In one embodiment of the present invention, wherein the cancer is breast cancer, pancreatic cancer, colorectal cancer, or gastric cancer.

In one embodiment of the present invention, wherein the radionuclide of the radiopharmaceutical is ¹³¹I, ¹⁷⁷Lu, ²¹¹At, ²²⁵Ac or ²²⁷Th.

In one embodiment of the present invention, wherein the radiopharmaceutical is a derivative of positron radioisotope-labeled prostate-specific membrane antigen (PSMA) inhibitor.

In one embodiment of the present invention, wherein the used positron radioisotope is ¹⁸F or ⁶⁸Ga.

The present invention also provides a method for treating cancer metastases, comprising administering a radiopharmaceutical to a subject; detecting the signal of the radiopharmaceutical in various organs in the subject by an instrument, when a signal of the radiopharmaceutical is detected in an organ indicates that the organ of the subject has early cancer metastases; and applying a surgery, a chemotherapy, a radiotherapy, an administration of drugs, or a combination thereof to the subject for early treatment of cancer metastases, wherein the subject is suffering from early cancer metastasis.

In addition, the present invention further provides a method for treating cancer hepatic metastases by using a derivative of a derived radionuclide-labeled prostate-specific membrane antigen (PSMA) inhibitor.

### Embodiments

The present invention is a method for early detecting cancer metastases by using a radiopharmaceutical, comprising the steps of administering a radiopharmaceutical to a subject; and detecting the signal of the radiopharmaceutical in various organs in the subject by an instrument, since the radiopharmaceutical will specifically bind to a target protein of cancer metastatic cells, when a signal of the radiopharmaceutical is detected in an organ indicates that the organ has early cancer metastases.

The present invention is also a method for treating cancer metastases, comprising administering a radiopharmaceutical to a subject; detecting the signal of the radiopharmaceutical in various organs in the subject by an instrument, when a signal of the radiopharmaceutical is detected in an organ indicates that the organ of the subject has early cancer metastases; and applying a surgery, a chemotherapy, a radiotherapy, an administration of drugs, or a combination thereof to the subject for early treatment of cancer metastases, wherein the subject is suffering from early cancer metastasis.

The following examples are not intended to be limiting, and are only used to present various aspects of the present invention.

### EXAMPLES

The mice were NOD.CB17-Prkdcscid/NcrCrlBltw (NOD-SCID) or NOD.Cg-PrkdcscidIl2rgtm1Wjl YckNarl (ASID), 4-6 weeks of age, purchased from the National Laboratory Animal Center of the National Applied Research Laboratories. Mice were anesthetized with 2% isoflurane, and breast cancer cells (MDA-MB-231.Luc and SKBR3) were injected into the lower end of the spleen of each mouse using a 30G needle, splenectomy and vascular ligation were performed after the spleen and blood vessels had turned white.

For establishing hepatic metastasis model, breast cancer cells (MDA-MB-231.Luc and SKBR3) were orthotopically injected into the ASID mice, at the same time, 1*10⁶ MDA-MB-231.Luc or 5*10⁶ SKBR3 cells were injected into the mammary fat pad of each mouse, Bal/C mice were prepared at the same time as an animal model of orthotopic, homologous mammary adenocarcinoma, and 1*10⁶ 4T1-Luc breast cancer cells were injected.

Generally, before the injection of the radiopharmaceutical [¹⁸F]PSMA-1007, the mice were continuously under anesthesia, and CT scan was used for correction, then 200-300 uCi (0.2 ml) of [¹⁸F]PSMA-1007 were injected into the tail vein of each mouse. The accumulations of the radiopharmaceutical were tracked through an Argus PET/CT scanner. In the PET/CT image, a standard uptake values (SUVs) were obtained and were used to establish a non-decay-corrected time-activity curve (TAC) of each organ, they could be used to estimate absorbed radiation doses and further to evaluate whether the absorbed radiation doses indicate early metastases or not.

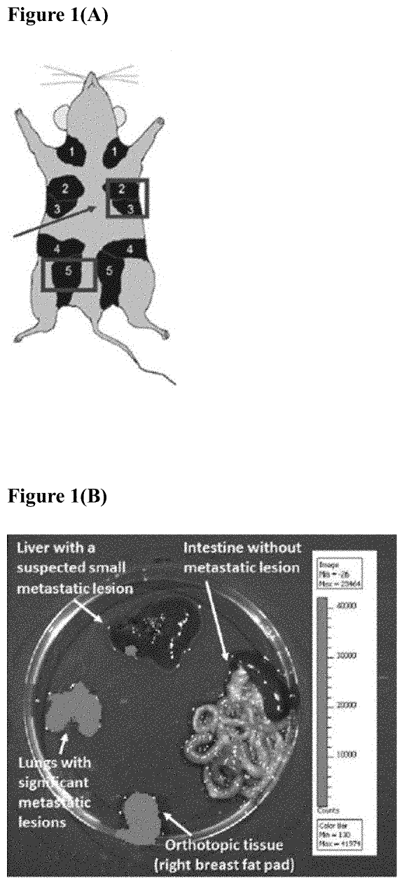

As shown in FIG. 1(A), Nos. 2, 3, and 5 were the locations of implanted tumor cells (in situ), and then through the non-invasive 3D in vivo molecular imaging system (Caliper IVIS Spectrum System, IVIS system). As shown in FIG. 1(B), metastases were found in the livers and lungs of the 4T1 tumor-bearing mice.

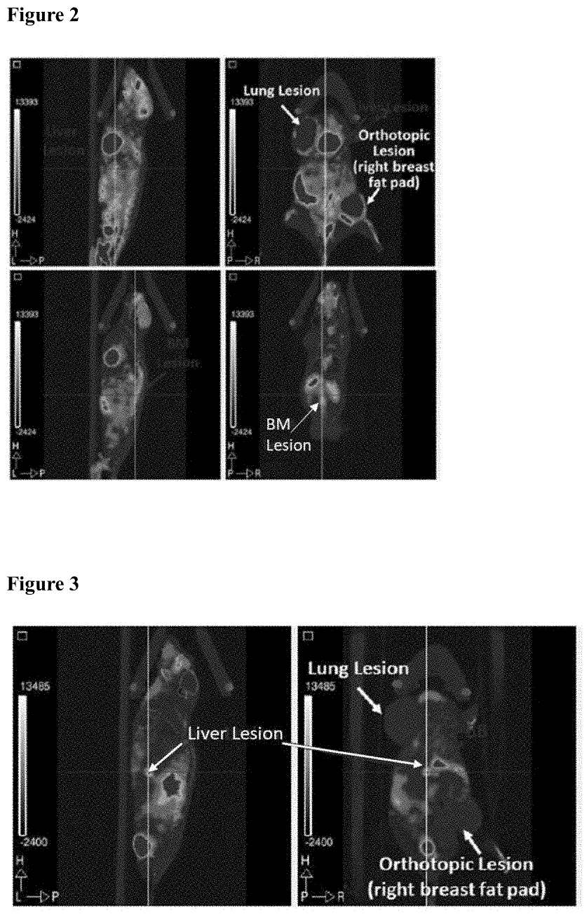

FIG. 2 indicates an observation of using [¹⁸F]FDG micro PET/CT imaging. After injection of [¹⁸F]FDG (200-300 uCi/0.2 ml) in animals, microPET imaging was started in the same mode for 90 minutes, and then PET data were collected according to the whole body emission protocol on two beds by the small animal Argus PET/CT scanner for 15 minutes, and breast cancer hepatic metastases and bone/lung metastatic cancer cells could be observed through this method. Based on the results, it was found that the results represented by the displayed images could not clearly distinguish the differences between the two.

FIG. 3 shows an observation of using ¹⁸F-PSMA-1007 as a radiopharmaceutical. After the drug was administered to 4T1-tumor mice, [¹⁸F]PSMA-1007 micro PET/CT imaging was used for observation. It was clearly observed that ¹⁸F-PSMA-1007 was specific and could be absorbed by breast cancer hepatic metastatic cells, but not by the primary breast cancer tumors and lung/bone metastatic tumor cells. Therefore, the effect of significant difference could be achieved in the image. It could be further used as a guide for therapeutic drugs, allowing the drugs with therapeutic functions to reach target sites, serving as an important tool for precise treatment.

The present invention has been described and illustrated in sufficient detail to enable those of ordinary skill in the art to which the present invention pertains to understand methods of making and using this art, however, various variations, modifications or improvements are possible and should be deemed to be no different from the spirit and scope of this invention. Those skilled in the art to which the present invention pertains can easily understand and realize the objects of the present invention and obtain the aforementioned results and advantages. The animals and instruments used in the present invention represent the best embodiment, are exemplary, and are not intended to limit the scope of the present invention. Those skilled in the art and the modifications or other uses that will occur when making or using this technology are all included in the spirit of the present invention and defined by the scope of rights.

What is claimed is:

1. A method for early detecting cancer metastases by using a radiopharmaceutical, comprising the steps:
administering a radiopharmaceutical to a subject; and
detecting the signal of the radiopharmaceutical in various organs in the subject by an instrument, when a signal of the radiopharmaceutical is detected in an organ indicates that the organ of the subject has early cancer metastases.

2. The method of claim 1, wherein the cancer metastases are cancer hepatic metastases.

3. The method of claim 1, wherein the cancer is breast cancer, pancreatic cancer, colorectal cancer or gastric cancer.

4. The method of claim 1, wherein the wherein the radionuclide of the radiopharmaceutical is ¹³¹I, ¹⁷⁷Lu, ²¹¹At, ²²⁵Ac or ²²⁷Th.

5. The method of claim 1, wherein the radiopharmaceutical is a derivative of positron radioisotope-labeled prostate-specific membrane antigen (PSMA) inhibitor.

6. The method of claim 5, wherein the positron radioisotope is ¹⁸F or ⁶⁸Ga.

7. A method for treating cancer metastases, which comprises administering a therapeutically effective dose of a radiopharmaceutical to a subject, comprising the steps of:
administering a radiopharmaceutical to a subject;
detecting the signal of the radiopharmaceutical in various organs in the subject by an instrument, when a signal of the radiopharmaceutical is detected in an organ indicates that the organ of the subject has early cancer metastases; and
applying a surgery, a chemotherapy, a radiotherapy, an administration of drugs, or a combination thereof to the subject for early treatment of cancer metastases,
wherein the subject is a subject suffers from early cancer metastasis confirmed by the detection method of claim 1.

8. The method of claim 7, wherein the cancer metastases are cancer hepatic metastases.

9. The method of claim 7, wherein the cancer is breast cancer, pancreatic cancer, colorectal cancer or gastric cancer.

10 .The method of claim 7, wherein the wherein the radionuclide of the radiopharmaceutical is ¹³¹I, ¹⁷⁷Lu, ²¹¹At, ²²⁵Ac or ²²⁷Th.

11. The method of claim 7, wherein the radiopharmaceutical is a derivative of positron radioisotope-labeled prostate-specific membrane antigen (PSMA) inhibitor.

12. The method of claim 11, wherein the positron radioisotope is ¹⁸F or ⁶⁸Ga.
